(19) United States
(12) Patent Application Publication
(10) Pub. No.: US 2022/0040351 A1
(43) Pub. Date: Feb. 10, 2022

(54) Disinfection apparatus and method

(71) Applicant: Lorenzo Lepore, Medford, MA (US)

(72) Inventor: Lorenzo Lepore, Medford, MA (US)

(21) Appl. No.: 17/505,538

(22) Filed: Oct. 19, 2021

Related U.S. Application Data
(63) Continuation-in-part of application No. 17/392,001, filed on Aug. 2, 2021.
(60) Provisional application No. 63/061,055, filed on Aug. 4, 2020.
(60) Provisional application No. 63/184,416, filed on May 5, 2021.

(51) Int. Cl. A61L 2/22

(52) U.S. Cl. CPC A61L 2/22; A61L 2202/11; A61L 2202/25; A61L 2202/16; A61L 2202/15

(57) ABSTRACT

Disinfecting apparatus and method. The disinfecting apparatus includes a first nozzle sized to be positioned with respect to a surface to disinfect the surface, a first hose configured to operably connect with the first nozzle, and a disinfectant generator. The disinfectant generator is configured to operably connect with the first hose and emit a disinfectant agent into the first hose so that the disinfectant agent travels through the first hose and exits the first nozzle to disinfectant the surface.

### CROSS REFERENCE TO RELATED APPLICATIONS

The present application is a continuation-in-part of U.S. patent application Ser. No. 17/392,001, filed on Aug. 2, 2021, which claims the benefit of and priority to U.S. provisional application No. 63/061,055, filed on Aug. 4, 2020, and co-pending U.S. provisional application No. 63/184,416, filed on May 5, 2021, the entire disclosures of each of which are incorporated by reference as if set forth in their entirety herein.

### TECHNICAL FIELD

Embodiments described herein generally relate to disinfectant devices and methods and, more particularly but not exclusively, to devices and methods for disinfecting surfaces.

### BACKGROUND

Musical instruments provide excellent environments for the growth of infectious microbes. Their design and intended use requires users to disperse oral and pulmonary fluids within the internal tubing or casing of instrument bodies. These fluids can remain in the instrument and serve as a culture for growing infectious microbes.

The recent COVID-19 pandemic has generated a heightened concern for microbial disease transmission. Disease transmission may occur through mucous membranes in the eyes, nose, or mouth. Wind instruments, for example, directly contact the mucous membranes of a user's mouth and come into close proximity with highly vulnerable pulmonary tissues. However, other contaminated instruments that only touch a user's skin still pose the risk of disease spread.

This is problematic in facilities such as schools, where multiple students use or handle the same instrument. Similarly, businesses such as music shops or pawn shops may only wipe external surfaces of instrument mouthpieces between different people using the instrument. As these instruments are not adequately disinfected, they can be a safety hazard to anyone who plays or handles the instrument.

Online marketplaces and ecommerce services also enable parties to exchange instruments, among other goods. These services allow people or organizations to exchange goods from essentially anywhere, including from foreign countries. These services also provide no safeguards or guarantees that a seller will take adequate safety measures before shipping an instrument to a buyer.

Also, other areas such as transportation vehicles (e.g., trains, busses, and aircraft) carry a large number of people and in a relatively small space. Passengers and crew contact several surfaces in these vehicles, and consequently these surfaces can be depository for microbes.

A need exists, therefore, for devices and methods for disinfecting instruments.

### SUMMARY

This summary is provided to introduce a selection of concepts in a simplified form that are further described below in the Detailed Description section. This summary is not intended to identify or exclude key features or essential features of the claimed subject matter, nor is it intended to be used as an aid in determining the scope of the claimed subject matter.

According to one aspect, embodiments relate to a disinfecting apparatus. The apparatus includes a first nozzle sized to be positioned with respect to a surface to disinfect the surface; a first hose configured to operably connect with the first nozzle; and a disinfectant generator configured to operably connect with the first hose and emit a disinfectant agent into the first hose so that the disinfectant agent travels through the first hose and exits the first nozzle to disinfect the surface.

In some embodiments, the first hose includes a plurality of apertures to emit the disinfectant agent from the first hose to disinfect the instrument.

In some embodiments, the disinfectant generator includes at least one ultrasonic transducer configured to volatize a disinfectant liquid to a fog or mist to create the disinfectant agent.

In some embodiments, the apparatus further includes at least a second hose configured to operably connect with the disinfectant generator to receive the disinfectant agent and emit the disinfectant agent to disinfect the surface. In some embodiments, the first hose is configured to disinfect a first surface, and the second hose is configured to disinfect a second surface at a same time the first hose disinfects the first surface.

In some embodiments, the apparatus further includes a vacuum device for removing moisture from the surface after the disinfectant agent has been emitted on the surface. In some embodiments, the vacuum device further comprises a vacuum adaptor to create a seal between the vacuum device and the surface.

In some embodiments, the disinfectant agent includes a disinfectant or a sanitizer.

In some embodiments, the apparatus further includes an exhaust force generator to generate an exhaust force on the surface to remove moisture from the surface.

In some embodiments, the first hose is configured to selectively connect with a second nozzle that is different than the first nozzle.

According to another aspect, embodiments relate to a method of manufacturing a disinfecting apparatus. The method includes connecting a first nozzle to a first hose, connecting the first hose to a disinfectant generator configured to hold a disinfectant agent, and providing a disinfectant emitter in operable connectivity with the disinfectant generator that is configured to emit the disinfectant agent through at least the first hose to disinfect a surface.

In some embodiments, the method further includes creating a plurality of apertures in the first hose that are configured to emit the disinfectant agent out of the first hose to disinfect the surface.

In some embodiments, the method further includes providing at least one ultrasonic transducer in the disinfectant generator to volatize a disinfectant liquid to a fog or mist to create the disinfectant agent.

In some embodiments, the method further includes providing the disinfectant generator with a vacuum device.

In some embodiments, the method further includes providing an adapter on the first hose to create a seal between the first hose and the surface.

According to yet another aspect, embodiments relate to a method for disinfecting a surface. The method includes operably positioning the first nozzle of the disinfecting apparatus described above, and activating the disinfectant generator described above to emit the disinfectant agent into or on the surface.

### BRIEF DESCRIPTION OF DRAWINGS

Non-limiting and non-exhaustive embodiments of the invention are described with reference to the following figures, wherein like reference numerals refer to like parts throughout the various views unless otherwise specified.

FIG. 1 illustrates a disinfecting apparatus in accordance with one embodiment;

FIG. 2 illustrates a diagram of the electronics of the disinfecting apparatus of FIG. 1 in accordance with one embodiment;

FIG. 3 illustrates a hose of the disinfecting apparatus of FIG. 1 in accordance with one embodiment;

FIG. 4 illustrates a disinfecting apparatus in accordance with another embodiment;

FIG. 5 illustrates a disinfecting apparatus in accordance with another embodiment;

FIG. 6 depicts a flowchart of a method of manufacturing a disinfecting apparatus in accordance with one embodiment; and

FIG. 7 illustrates user disinfecting surfaces in an airplane cabin in accordance with one embodiment.

### DETAILED DESCRIPTION

Various embodiments are described more fully below with reference to the accompanying drawings, which form a part hereof, and which show specific exemplary embodiments. However, the concepts of the present disclosure may be implemented in many different forms and should not be construed as limited to the embodiments set forth herein; rather, these embodiments are provided as part of a thorough and complete disclosure, to fully convey the scope of the concepts, techniques and implementations of the present disclosure to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

As discussed above, musical instruments provide excellent environments for the growth of infectious microbes. In schools, for example, multiple students may use the same instrument as part of their music classes, and these instruments are rarely disinfected between uses. For example, a school's disinfectant procedure may involve only wiping the mouthpieces of instruments between uses. Schools may occasionally send instruments out for a more thorough cleaning or disinfection procedure, but this tends to occur infrequently and still does not adequately disinfect the instruments.

There is also no way to ensure the health of all instrument users. Accordingly, students may be at risk of passing along and being exposed to harmful microbes from other, possibly ill students.

Numerous peer reviewed and published medical studies have demonstrated that wind instruments in particular enable the growth of infectious microbes because of their internal tubing. These instruments also directly contact the mucous membranes of a user's mouth, which are in close proximity to highly vulnerable pulmonary tissues. However, even other instruments that only touch a user's skin still pose a health risk to users.

Instruments are often stored in instrument cases for an extended period of time as well. Students may go several days without removing their instrument from the instrument's case. The dark, moist environment of the case's interior is also conducive to microbe growth.

Embodiments described herein provide devices and methods for disinfecting instruments or other devices or surfaces. The term “disinfect” as used in the present application may refer to the process of removing microbes or microorganisms such as bacterium or viruses (for simplicity, “microbes”). In the context of the present application, “microbes” may also refer to substances that could cause disease or otherwise bring harm to a person if the person were exposed to said substances.

The term “disinfect” recognizes that not all microbes may be removed by the embodiments herein. For example, some instruments such as French horns have up to 18 feet of tubing and a disinfectant agent may not “reach” all internal surfaces thereof in casual use. Accordingly, the disinfectant agent described below may not contact every surface of an instrument to remove all microbes from the instrument but will still greatly minimize the risk of microbial transmission.

Even if the disinfectant agent reaches a particular surface, the agent may not remove 100% of the microbes from the surface. Accordingly, “disinfect” may refer to the process of removing at least some amount of microbes such that a person is less likely to be exposed to microbes had the disinfectant process not been performed.

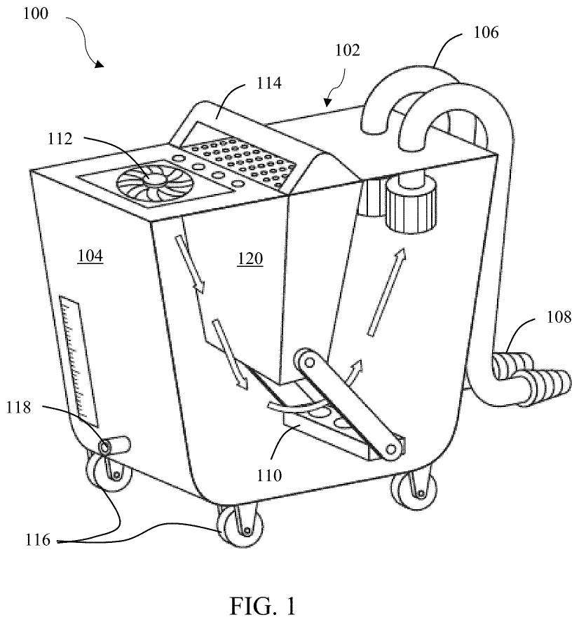

The embodiments of the present application provide devices and methods for disinfecting a musical instrument. FIG. 1 illustrates a disinfecting apparatus 100 in accordance with one embodiment. The apparatus 100 may include a disinfectant generator 102 with a tank 104, one or more hoses 106, and one or more nozzles or adaptors 108.

The disinfectant generator 102 may include one or more ultrasonic transducers 110 in the tank 104 to convert a liquid (not shown in FIG. 1) to a mist or fog to act as a disinfectant agent. The disinfectant generator 102 may include a pressure fan 112 to force the disinfectant agent through the hose(s) 106 and into an instrument (also not shown in FIG. 1).

The disinfectant generator 102 may further include, for example, a handle 114 to facilitate carrying of the disinfectant generator 102, wheels 116 to facilitate movement of the generator 102, and a drain 118 to remove liquid from the tank 104. The disinfectant generator 102 may also include any required electronics 120 to power the apparatus 100 and control the operation of components thereof.

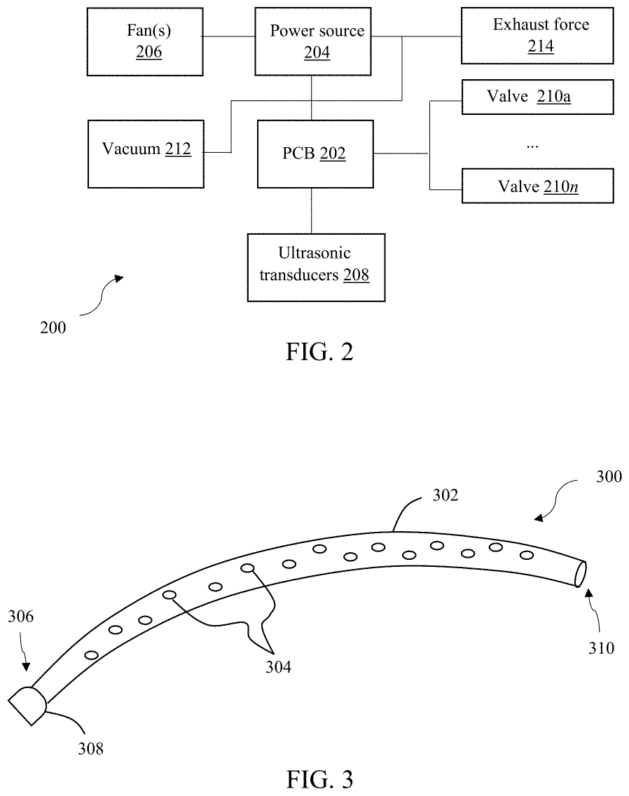

FIG. 2 illustrates an electrical diagram 200 of the electronics 120 of FIG. 1. The electronics 120 may include a printed circuit board (for simplicity, “PCB”) 202 in connection with a power source 204. The PCB 202 may be in operable communication with and issue instructions to one or more fan controllers 206, ultrasonic transducer(s) 208, and valves 210a-n where n is the number of valves configured with the disinfectant generator. For example, there may a valve associated with each hose extending from the disinfectant generator. The PCB 202 may instruct a particular valve 210 to open to allow the disinfectant agent to flow through its associated hose, or to close to prevent the disinfectant agent from flowing through its associated hose.

The PCB 202 and power source 204 may instruct and supply power to the ultrasonic transducer(s) 208. For example, upon activation, the ultrasonic transducer(s) 208 may vibrate or create waves to volatilize the liquid agent in the disinfectant generator into the disinfectant agent.

Some embodiments of the disinfectant generator may use an ionized hydrogen peroxide process to disinfect an instrument. For example, some embodiments may apply Binary Ionization Technology™ to a low-percentage Hydrogen Peroxide(H₂O₂)-based solution to create an activated ionized hydrogen peroxide (for simplicity, “AIHP”) fog or mist that is applied to surfaces of an instrument to disinfect the instrument. After exposure to cold plasma activation, the hydrogen peroxide is converted to OH ions such as hydroxyl radicals, which are a type of reactive oxygen species. This reactive oxygen species damages pathogenic organisms through oxidation of proteins, carbohydrates, and lipids. This damage leads to cellular disruptions or dysfunctions, thereby disinfecting targeted surfaces.

This chemical process is only exemplary and does not limit the disinfectant agent to a particular substance or process. Other types of disinfectant agents may be used as long as they can disinfect surfaces to accomplish the objectives of the embodiments described herein.

The PCB 202 may also control the amount of power supplied from the power source 204 to the various components of the disinfectant generator. For example, if multiple valves are open, the fan(s) may require more power to adequately emit the disinfectant agent through multiple hoses. If the disinfectant generator has only one valve (or only one valve that is open), the power source 204 may supply less power to the fans to adequately emit the disinfectant agent through the hose and conserve power.

Similarly, different instruments may require different pressure drives. This may be due to the size of the instrument or the anatomy of the instrument. For example, a straight tube instrument such as a clarinet or a flute may require a less intense pressure drive than that of a French horn or other type of instrument with narrow and convoluted pathways from the mouthpiece to the bell.

With multiple hoses, the disinfectant generator may accommodate multiple instruments or surfaces per disinfectant application or cycle. In some embodiments, the disinfection capacity would be limited only by the size of the tank that holds the liquid disinfectant.

In some embodiments, multiple hoses with adaptors may service different parts of a single instrument. For example, a hose with one adaptor may disinfect the bell of an instrument while another hose with a different adaptor may simultaneously disinfect the mouthpiece of the instrument.

In some embodiments, the disinfectant generator may further include a vacuum device 212 for removing moisture remaining from the disinfectant process. For example, the vacuum device 212 may comprise an output adaptor that is shaped and sized to fit in the bell of an instrument to create a seal therewith. During a vacuum operation, a vacuum device 212 such as a Venturi vacuum generator cartridge may draw in air through an output adaptor and, as a result, remove any moisture from the instrument. In some embodiments, the output adapter may comprise an indicator to validate that the disinfectant agent has passed through the instrument or that the instrument interior is dry.

In some embodiments, the PCB 202 and the power source 204 may also be in operable connectivity with an exhaust force generator 214. The exhaust force generator 214 may generate an exhaust force through the instrument to remove moisture from the instrument. For example, the disinfectant generator may include any required blowers, pumps, tubing, or adaptors to force pressurized air through the instrument.

Referring back to FIG. 1, in operation the hose 106 may be operably positioned with respect to an instrument, such as inserted into a portion of the instrument. The disinfectant agent generated by the disinfectant generator 102 may be forced out of the tank 104, through the hose 106, and into the instrument to disinfect the instrument. For example, the hose 106 may be inserted into the bell of an instrument. To achieve a desired fit with respect to the instrument, a nozzle 108 that conforms to the shape of the instrument body (e.g., the inner surface of an instrument's bell) may be inserted into the instrument.

FIG. 3 illustrates a hose 300 in accordance with one embodiment. The hose 300 may be used in conjunction with the disinfectant generator 102 of FIG. 1. The hose 300 may include a tube portion 302 with one or more apertures 304 such as holes or slits that allow a disinfectant agent to be emitted from the tube portion 302.

In operation, the proximal end 306 may be operably connected to a disinfectant generator such as the disinfectant generator 102 of FIG. 1 via a connection mechanism 308. The distal end 310 may be inserted into various locations of an instrument body or otherwise operably positioned with respect to an instrument. Upon activation, the disinfectant generator may deliver the disinfectant agent to the hose 300 such that the disinfectant agent travels through at least a portion of the tube portion 302 and out of the aperture(s) 304 or the distal end 310.

The tube portion 302 may comprise any type of material that can carry the disinfectant agent and is flexible enough to fit into an instrument. The tube portion 302 may have a customized length and width depending on the type of instrument it is intended to disinfect. For example, a thicker tube portion 302 may be used to disinfect a tuba than one used to disinfect a piccolo.

The one or more apertures 304 may allow the emission of the disinfectant agent during the disinfectant process. In some embodiments, the one or more apertures 304 may be evenly distributed along the length of the tube portion 302. In some embodiments, however, the tube portion 302 may not have any apertures 304.

The connection mechanism 308 may connect the tube portion 302 with the disinfectant generator. For example, the connection mechanism 308 may comprise a rubber plug that can deform and be placed into and seal a hole of the tank 104 by an interference or pressure fit. Alternatively, the connection mechanism 308 may include a series of threads or slots to engage a respective surface of the disinfectant generator 102.

In some embodiments, such as in FIG. 1, the hose 106 may further include a nozzle or adaptor (for simplicity, “nozzle”) 108 that is configured to fit inside of an instrument body or otherwise engage an instrument. For example, the nozzle 108 may be placed within the lumen of an instrument body to create a seal therewith. The nozzle 108 may be customized to fit into a specific type of instrument, such as a specific type of wind instrument.

These nozzles may be readily exchangeable such that a user can remove a first nozzle 108 from the hose 106 and attach a second, differently-sized or shaped nozzle. For example, a nozzle for a flute may be smaller than a nozzle for a saxophone. Different nozzles may be used for the bell and joints of an instrument.

The nozzle 108 may operably connect with the hose 106 via a pressure or interference fit. Alternatively, the nozzle 108 may include a threaded portion to engage a threaded portion of the hose 106. The exact configuration of the nozzle(s) 108 and how they connect with the hose 106 may vary.

In some embodiments, the hose 106 may alternatively be configured with an adaptor. Rather than, for example, a nozzle being inserted into the lumen of an instrument, a nozzle may be configured to create a seal around one end of the instrument to drive the disinfectant agent through the instrument. For example and without limitation, the adaptor may be formed of a rubber material and configured to create a seal around the mouthpiece of an instrument.

In some embodiments, a user may create and use a mold of the mouthpiece so that the nozzle creates a seal around the instrument mouthpiece. In some embodiments, the mold may attach to the outside of the instrument.

The ultrasonic transducer(s) 110 may be activated to volatilize the liquid agent. The ultrasonic transducer(s) may comprise one or more metal plates that may, upon activation, vibrate at such frequencies to convert the liquid agent into a fog, mist, or otherwise to a gaseous state to act as the disinfectant agent. The disinfectant agent may look like smoke and provide a visual validation that the disinfectant agent has penetrated the instrument or a component thereof.

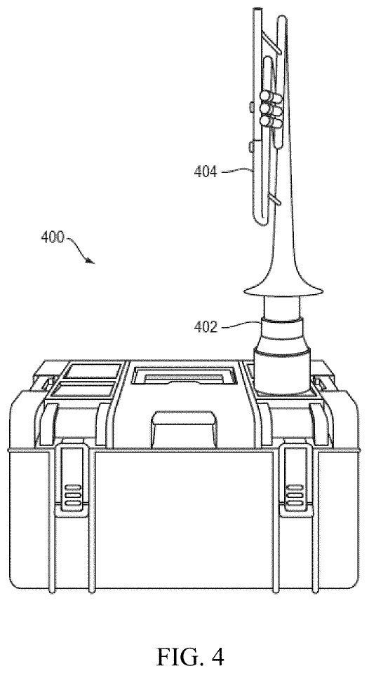

The configuration of the instrument disinfecting apparatus 100 of FIG. 1 is merely exemplary, and the instrument disinfecting apparatus may be configured in a variety of ways. For example, FIG. 4 depicts a case 400 that may be used to transport or protect a disinfectant generator such as the disinfectant generator 102 of FIG. 1. In this embodiment, a hose may be positioned vertically and connected to a vertical adaptor or nozzle 402. In this configuration, an instrument such as a trumpet 404 may be positioned over the nozzle 402 to undergo the disinfectant process.

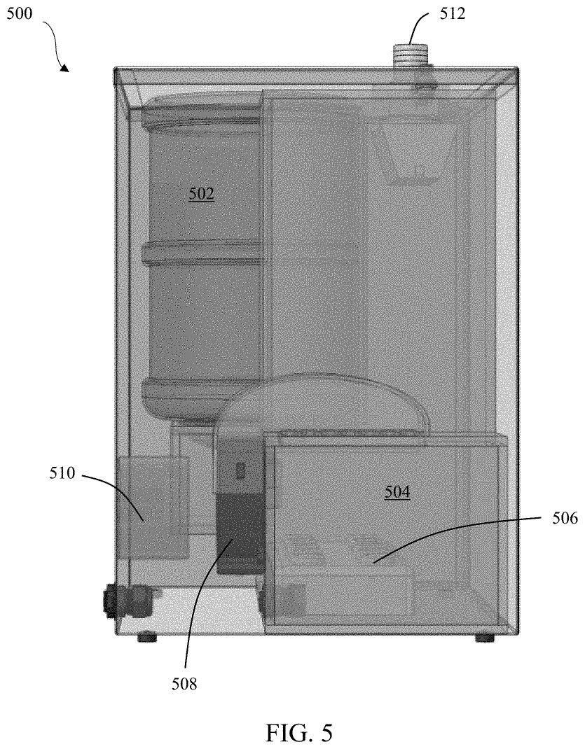

As another example, FIG. 5 illustrates a front view of a disinfectant generator 500 in accordance with another embodiment. The disinfectant generator 500 of FIG. 5 may include a reservoir 502 to supply a liquid disinfectant to a tank 504. A mist generator 506 such as one or more ultrasonic transducers may be in operable connectivity with the tank 504 such that it can convert liquid therein to a mist. The mist generator 506 may be configured similarly to the ultrasonic transducers 110 of FIG. 1, for example.

One or more fans 508 operably positioned with respect to a fan intake 510 may force the disinfectant agent (in mist form) through the disinfectant generator 500 and out of the outlet 512. Although not shown in FIG. 5, the disinfectant generator 500 may receive at the outlet 512 a hose such as the hose 300 of FIG. 3.

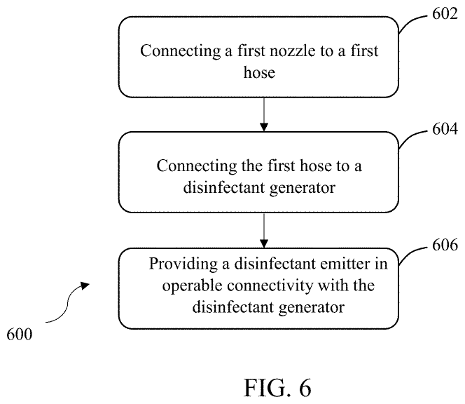

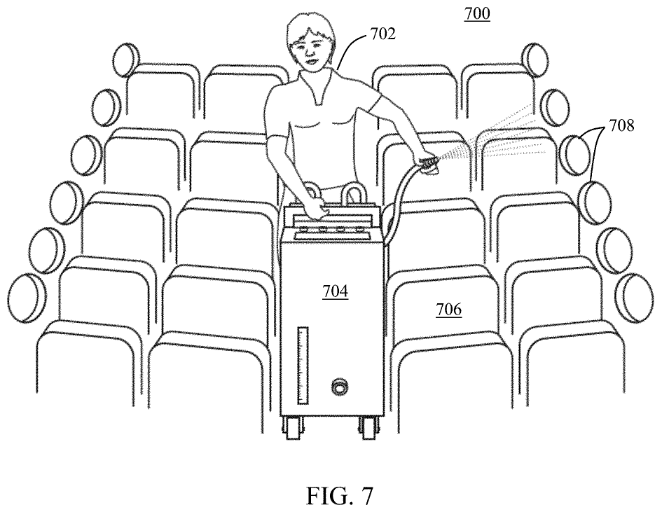

FIG. 6 depicts a flowchart of a method 600 for manufacturing an instrument disinfecting apparatus in accordance with one embodiment. Step 602 involves connecting a first nozzle to a first hose. The hose may be similar to the hose of FIGS. 1 and 3, for example, and may comprise a flexible tube portion that can be inserted into an instrument. Step 602 may similarly involve connecting an adaptor to the first hose in lieu of a nozzle. The nozzle or adaptor may operably connect with the first hose via a pressure or interference fit, for example.

Step 604 involves connecting the first hose to a disinfectant generator configured to hold a disinfectant agent. The disinfectant generator may be similar to the disinfectant generator 100 of FIG. 1, for example. The disinfectant generator may be configured to hold a disinfectant such as a liquid disinfectant that can be volatilized into a mist.

Step 606 involves providing a disinfectant emitter in operable connectivity with the disinfectant generator that is configured to emit the disinfectant agent through at least the first hose to disinfect an instrument. For example, the disinfectant generator may include one or more fans to help propel the disinfectant agent out of the disinfectant generator to the instrument.

Method 600 may include additional steps related to manufacturing the disinfectant generator. For example, the method may further include providing at least one ultrasonic transducer in the disinfectant generator to volatize a disinfectant liquid to a fog or mist to create the disinfectant agent, providing the disinfectant generator with a vacuum device, and creating a plurality of apertures in the first hose that are configured to emit the disinfectant agent out of the first hose to disinfect the instrument.

Although the present application is largely described in the context of disinfecting musical instruments, the embodiments described herein may be used in a variety of other applications. For example, the embodiments herein may disinfect furniture surfaces, medical equipment, sporting equipment, surfaces in transportation vehicles, tools, breathalyzers, drinkware, foodware, cookware, or the like. The above list is not exhaustive, and the embodiments described herein may disinfect other structures or devices in addition to or in lieu of those listed above.

As a more specific example of transportation vehicles, the embodiments described herein may disinfect surfaces on aircraft such as airplanes. FIG. 7 illustrates the interior of an airplane cabin 700 in which a user 702 uses a disinfecting apparatus 704 to disinfect surfaces such as those on seats 706 or windows 708. The disinfecting apparatus 704 may be similar to the disinfecting apparatus 100 of FIG. 1.

Commercial airplanes transport over one million passengers per day. Often times a single flight transports hundreds of passengers, none of whom are able to socially distance during the flight.

Although airplanes are equipped with air-filtration technologies, there is still the potential for passengers to transmit microbes onto surfaces in the airplane and to be exposed to microbes on the airplane. These surfaces may include, but are not limited to, armrests, seats, headrests, electrical outlets, tray tables, tray table locks, windows, window covers, safety pamphlets or other reading materials, fan controls, light controls, overhead storage covers, overhead storage cover handles, lavatory doors, lavatory door handles, surfaces within a lavatory, etc. Accordingly, there are plenty of surfaces to disinfect between flights.

There is typically a short window of time between when a plane is scheduled to arrive at a gate to disembark passengers, and when other passengers are scheduled to board the plane for a subsequent flight. As a result these surfaces must be disinfected as quickly as possible.

The embodiments herein may be used in conjunction with a disinfectant process to disinfect airplane surfaces. These surfaces may include those listed above, for example. In these applications, the apparatus may be held, carried, or rolled (as in FIG. 7) by a worker to emit the disinfectant on any required surfaces in the airplane. The apparatus may use any combination of tube portions, nozzles, disinfectant generators, connection mechanisms, or the like.

As another exemplary application, the embodiments described herein may disinfect surfaces in healthcare institutions such as hospitals, clinics, urgent care facilities, intensive care units, doctor's offices, or other types of locations associated with various healthcare providers. Healthcare institutions such as these treat patients, some of whom may be suffering from one or more diseases. Throughout the course of treatment, these patients and other personnel may transmit microbes onto various surfaces or objects and be exposed to microbes therefrom. This problem can be exacerbated if patients are carriers of harmful diseases.

Accordingly, healthcare institutions may face heightened pressure to disinfect various surfaces therein. These surfaces may include, but are not limited to, beds, pillows, chairs, tables, armrests, doors, door handles or knobs, nightstands, elevators, elevator buttons, kiosks, touchscreens, restrooms, trash cans, hallways, diagnostic equipment, monitoring equipment, or the like.

In these applications, the apparatus may be held, carried, or rolled by a user as the user moves through the healthcare institution and disinfects surfaces therein. The apparatus may use any combination of tube portions, nozzles, disinfectant generators, connection mechanisms, or the like.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, or alternatively, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Having described several example configurations, various modifications, alternative constructions, and equivalents may be used without departing from the spirit of the disclosure. For example, the above elements may be components of a larger system, wherein other rules may take precedence over or otherwise modify the application of various implementations or techniques of the present disclosure. Also, a number of steps may be undertaken before, during, or after the above elements are considered.

Having been provided with the description and illustration of the present application, one skilled in the art may envision variations, modifications, and alternate embodiments falling within the general inventive concept discussed in this application that do not depart from the scope of the following claims.

What is claimed is:

1. A disinfecting apparatus comprising:
a first nozzle sized to be positioned with respect to a surface to disinfect the surface;
a first hose configured to operably connect with the first nozzle; and
a disinfectant generator configured to:
operably connect with the first hose, and
emit a disinfectant agent into the first hose so that the disinfectant agent travels through the first hose and exits the first nozzle to disinfect the surface.

2. The apparatus of claim 1, wherein the first hose includes a plurality of apertures to emit the disinfectant agent from the first hose to disinfect the instrument.

3. The apparatus of claim 1 wherein the disinfectant generator includes at least one ultrasonic transducer configured to volatize a disinfectant liquid to a fog or mist to create the disinfectant agent.

4. The apparatus of claim 1 further comprising at least a second hose configured to:
operably connect with the disinfectant generator to receive the disinfectant agent, and
emit the disinfectant agent to disinfect the surface.

5. The apparatus of claim 4 wherein the first hose is configured to disinfect a first surface, and the second hose is configured to disinfect a second surface at a same time the first hose disinfects the first surface.

6. The apparatus of claim 1 further comprising a vacuum device for removing moisture from the surface after the disinfectant agent has been emitted on the surface.

7. The apparatus of claim 6 wherein the vacuum device further comprises a vacuum adaptor to create a seal between the vacuum device and the surface.

8. The apparatus of claim 1 wherein the disinfectant agent includes a disinfectant or a sanitizer.

9. The apparatus of claim 1 further comprising an exhaust force generator to generate an exhaust force on the surface to remove moisture from the surface.

10. The apparatus of claim 1 wherein the first hose is configured to selectively connect with a second nozzle that is different than the first nozzle.

11. A method of manufacturing a disinfecting apparatus, the method comprising:
connecting a first nozzle to a first hose;
connecting the first hose to a disinfectant generator configured to hold a disinfectant agent;
providing a disinfectant emitter in operable connectivity with the disinfectant generator that is configured to emit the disinfectant agent through at least the first hose to disinfect a surface.

12. The method of claim 11 further comprising creating a plurality of apertures in the first hose that are configured to emit the disinfectant agent out of the first hose to disinfect the surface.

13. The method of claim 11 further comprising providing at least one ultrasonic transducer in the disinfectant generator to volatize a disinfectant liquid to a fog or mist to create the disinfectant agent.

14. The method of claim 11 further comprising providing the disinfectant generator with a vacuum device.

15. The method of claim 14 further comprising providing an adapter on the first hose to create a seal between the first hose and the surface.

16. A method for disinfecting a surface, the method comprising:
operably positioning the first nozzle of the disinfecting apparatus of claim 1 with respect to a surface; and
activating the disinfectant generator of claim 1 to emit the disinfectant agent into or on the surface.
